(19) United States
(12) Patent Application Publication
(10) Pub. No.: US 2026/0207262 A1
(43) Pub. Date: Jul. 23, 2026

(54) Wide Angle Navigation System

(71) Applicant: Stryker European Operations Limited, Carrigtwohill (IE)

(72) Inventor: Ulrich Hoffmann, Breisach (DE)

(21) Appl. No.: 19/138,726

(22) PCT Filed: Dec. 13, 2022
(86) PCT No.: PCT/EP2022/085678

§ 371 Date: Jun. 13, 2025

(51) Int. Cl. A61B 34/20; A61B 6/00; A61B 90/00; A61B 90/50; G06T 7/246; G06T 7/73

(52) U.S. Cl. CPC A61B 34/20; A61B 6/547; G06T 7/248; G06T 7/74; A61B 2034/2057; A61B 2034/2065; A61B 2090/365; A61B 2090/376; A61B 2090/3937; A61B 2090/502; G06T 2207/10116; G06T 2207/30004; G06T 2207/30204

(57) ABSTRACT

Navigation system and corresponding method for operating a navigation system for intraoperatively tracking objects under surgery improving exactness of determination of spatial position and orientation of objects to be tracked with respect to each other.

### FILED OF THE INVENTION

The present invention relates to a navigation system and a navigation method for computer assisted surgery CAS, and in particular to a navigation system and a navigation method, which provides an increased accuracy and repeatability of procedures, increases confidence of surgeons, and reduces x-ray radiation.

### BACKGROUND OF THE INVENTION

Surgical procedures have improved over the recent years. Significant improvements have been achieved by systems for supporting the clinical personal, in particular surgeons, during surgeries. In particular bone fractures benefit from supporting systems for surgeons, which allows the surgeon to improve exactness of repositioning of bone parts and positioning of implants, like screws, nails, and bone plates, as well as tools and targeting and guiding devices.

As traumatized bones, i.e., fractures, have only a limited visual access, monitoring is usually based on radiating principles, like X-ray imaging or computer tomography CT images, or magnet resonance tomography MRT images. All these principles and methods involve at least one of the drawbacks of being radiation intensive, requiring large devices and requiring a considerable amount of time. Each monitoring step during a surgery prolongs the surgery duration and thus the duration of anesthesia and increases costs and radiation impact.

Therefore, there is a need for a navigation system and a corresponding method, which reduce imaging effort and thus duration of the surgery, reduce radiation impact on the patient and the surgeon, but at the same time maintain or increase the level of exactness of the surgery.

### SUMMARY OF THE INVENTION

The present invention provides a navigation system for computer assisted surgery (CAS) and a method for navigating within a computer assisted surgery (CAS) according to the independent claims, whereas further embodiments are incorporated in the dependent claims.

According to an exemplary embodiment, there is provided a navigation system for intraoperatively tracking objects during surgery, comprising an optical imaging system with a first optical imaging unit with a first optical imaging area and a second optical imaging unit with a second optical imaging area, and with a mechanical interface for coupling the imaging system to a reference object within a surgical environment; a first object tracking unit, e.g. an x-ray imaging device tracking unit, to be coupled to the first object, e.g. an x-ray imaging device, representing a spatial position and orientation of the first object, e.g. the x-ray imaging device, within a surgical environment; a second object tracking unit to be coupled to a second object, representing a spatial position and orientation of the second object within a surgical environment; and an image processing unit; wherein the first optical imaging unit and the second optical imaging unit are arranged with respect to each other such that the first optical imaging area and the second optical imaging area form an optical imaging area; wherein the first object tracking unit, e.g. the x-ray imaging device tracking unit, comprises a unique optical pattern allowing determining a spatial position and orientation of the first object, e.g. the x-ray imaging device, upon optical imaging with respect to the imaging system; wherein the second object tracking unit comprises a unique optical pattern allowing determining a spatial position and orientation of the second object upon optical imaging with respect to the imaging system; wherein the optical imaging area covering at least the unique optical pattern of the first object tracking unit, e.g. the x-ray imaging device tracking unit and the unique optical pattern of the second object tracking unit; wherein the imaging processing unit is adapted to determine the relative spatial position and orientation of the first object tracking unit, e.g. the x-ray imaging device tracking unit, and of the second object tracking unit with respect to each other based on optical images taken by the optical imaging system.

Thus, imaging may take place within the surgical environment without the need for an external imaging device in the surgical surrounding. Surgical environment is to be understood as the immediate environment where the surgery takes place, e.g., where the patient is located. Relative to the surgical environment the surgical surrounding is to be understood as the space where all surgical periphery is located. This may be for example the room in a hospital where the surgery takes place. Placing the imaging system within the surgical environment, e.g., coupled to a patient's body or a table where the patient is positioned, allows imaging of the relevant objects or components provided with unique optical patterns representing position and orientation of said respective object or component. An optical pattern or unique optical pattern may be a pattern visible in a spectral frequency range, which is visible for a human being. An optical pattern or unique optical pattern may also be a set of infrared IR lights or IR reflective markers, e.g., IR-LED's having a unique arrangement allowing determination of its spatial position and orientation. The optical imaging system then is IR-sensitive in order to image the optical IR pattern. A unique optical pattern is designed to allow determination of its spatial position and orientation. A unique optical pattern may also carry other information, such as for example information for identifying the object to be tracked to which this respective unique optical pattern is mounted. The imaging system coupled to a reference object thus may determine the relative position and orientation of the objects with respect to each other, including the relative position and orientation of the reference object, where the optical imaging system is mounted on. A reference object, in particular a reference object within a surgical environment is the object to which the other objects are set into a spatial relation. The reference object may be fixed to an inventory of the surgical environment, like an operating table or a support structure. The reference object may also be fixed to a patient's anatomy. Fixing to a patient's anatomy may directly provide a reference to the patient's anatomy without the need for an additional object representing the patient's anatomy to be tracked. The number of objects to be tracked and each provided with a unique optical pattern is generally not limited. Imaging from the within the surgical environment strongly reduces the probability for line-of-sight problems, i.e., the probability that a surgeon or other OR personnel block the line-of-sight between imaging device and optical pattern is reduced. Imaging several objects within a surgical environment each provided with a unique optical pattern with an imaging system provided also within this surgical environment requires a wide imaging area, which may be achieved by a wide-angle optical lens or a combination of more than one camera system with abutting or at least overlapping imaging area. A first optical imaging unit with a first optical imaging area and a second optical imaging unit with a second optical imaging area allows a wide optical imaging area which may be composed of abutting or overlapping imaging areas of two or even a larger number of imaging units, so as to form a continuous optical imaging area. The number of imaging units is not limited. With this respect, it is of relevance that the imaged objects with their unique optical patterns fall within an optical imaging area, which may be a continuous optical imaging area. A continuous optical imaging area is an optical imaging area without separation between optical imaging sub-areas. In case of a single optical lens, its optical imaging area usually is continuous. If two or more optical lenses are used, the total optical imaging area thereof is continuous if the optical sub-areas, each allocated to a single of the two or more optical lenses overlap to form a connected or overlapping total optical imaging area. The continuous area is required in order to have no gaps, which may hide or obscure a misalignment of the optical sub-areas. If the optical sub-areas abut or overlap, an image processing may establish a smooth transit and alignment between the two or more optical sub-areas. The continuity of a continuous optical area may also be achieved by providing a well-known reference pattern with a unique first reference sub-pattern laying in a first optical imaging sub-area and a unique second reference sub-pattern laying in a second optical imaging sub-area. The same applies for three or more, where a reference pattern has three or respectively more unique reference sub-patterns laying in a respective one of the three or more optical imaging sub-areas. As an alternative or in addition, calibration of a spatial position of the first optical imaging sub-area and the second (and any further) optical imaging sub-area with respect to each other may be carried out during manufacturing of an optical imaging system. As an alternative or in addition, calibration of a spatial position of the first optical imaging sub-area and the second (and any further) optical imaging sub-area with respect to each other may be carried out before an operation, e.g., by applying an optical reference pattern as described above, which after calibration can be removed.

According to a further embodiment, the navigation system comprises one or more further object tracking units, e.g., a third, a fourth and so on object tracking unit, to be coupled to a respective further object, representing a spatial position and orientation of the respective further object within a surgical environment, wherein the respective further object tracking unit comprises a unique optical pattern allowing determining a spatial position and orientation of the respective further object upon optical imaging with respect to the imaging system, wherein the continuous optical imaging area also covering the respective further unique optical pattern of the respective further object tracking unit, wherein the imaging processing unit is adapted to determine the relative spatial position and orientation of said object tracking units with respect to each other based on optical images taken by the optical imaging system.

Thus, a plurality of objects, including but not limited to components of an x-ray imaging system like a c-arm, an anatomy of a patient, even more anatomy objects, e.g., fragments of a fracture, one or more tools and implants or the like, may be determined with respect to their spatial position and orientation with respect to each other.

According to a further embodiment the first optical imaging unit and the second optical imaging unit each have a camera with a lens having an opening angle of at least 180 degrees (half sphere), wherein the first optical imaging unit and the second optical imaging unit are arranged back-to-back so that the first optical imaging area and the second optical imaging area together form the optical imaging area of 360 degrees (full sphere).

Thus, a total sphere imaging area may be provided by two imaging units mounted back-to-back. In case each of the imaging units has an optical imaging area of at least 180 degrees, i.e., a half sphere, and also having opposite viewing direction, the borders of the imaging area of both imaging units may abut or overlap along the entire circumference, thus forming a full sphere total optical imaging area. This allows an arbitrary positioning within the surgical environment, as long as no other obstacles are in the viewing directions toward the objects to be tracked or the unique optical patterns thereof.

According to a further embodiment the mechanical interface of the optical imaging system is adapted for establishing a reproducible spatial position and orientation of the optical imaging system with respect to the reference object within the surgical environment.

Thus, a reproducible spatial position and orientation of the imaging system and the reference object allows determining the defined relative spatial position and orientation of the reference object with respect to the objects having unique optical patterns. The reference object does not need a unique optical pattern, as long as it carries the optical imaging system. Nevertheless, the reference object may also be provided with a unique optical pattern in case the imaging system is mounted to an alternative reference object or if two or more optical imaging systems are used.

According to a further embodiment, the image processing unit is adapted to determine the relative spatial position and orientation of the reference object, the first object tracking unit, e.g., an x-ray imaging device tracking unit, and of the second object tracking unit with respect to each other based on optical images taken by the optical imaging system.

Thus, it is possible not only determining the relative spatial position and orientation of the first and second object with respect to each other, but also the relative spatial position and orientation of the reference object, as well as the first and second object with respect to each other.

According to a further embodiment, the optical imaging system comprises a housing, wherein the housing comprises the mechanical interface for coupling the optical imaging system to the reference object within a surgical environment, wherein the housing comprises an optical image unit receiving interface for interchangeably receiving at least one optical imaging unit of the optical imaging system for establishing a reproducible spatial position and orientation of the at least one optical imaging unit with respect to the optical imaging system.

Thus, it is possible to provide a housing where the imaging units may be exchanged. The housing may be designed as a single use housing, whereas the imaging units may be reused. The housing may be kept sterile and may provide a safe barrier to the exchangeable imaging units, which may not be sterilized. Optical windows or lenses or at least part of the lens arrangement may be provided at the housing side in order to be able to provide a closed barrier, whereas other parts of the lens arrangement of the optical units may be provided at the imaging units'side to be re-usable.

According to a further embodiment, the housing is a single use housing comprising deformable housing sections, which deformable housing sections are adapted to modify essential housing parts upon a sterilization process, such that a reuse is prevented upon application of a sterilization process.

Thus, it may be avoided that a housing may be re-used. If against the intention to use the housing only once, the housing is sterilized, the housing may deform, which deformation may also include a change of essential properties required for operating the optical system. This may also include clouding of windows or optical lenses provided at the housing side to avoid re-use of the housing. The change may be generated by different impacts each resulting from a sterilization. Essential housing parts in general are parts of the housing, which are relevant for the operation of the navigation system and the imaging system, respectively. Modifying essential housing parts may include deformation of particular housing portions or the entire housing, change a color of the housing, obscuring optical elements like clouding lenses or windows, so that no operation is possible any longer.

According to a further embodiment, the housing is adapted for receiving a cordless power supply of the optical imaging system.

Thus, the optical imaging system may be operated autarkic with respect to power supply without the need for a power cable connection within the surgical environment.

According to a further embodiment, the housing is adapted for receiving a wireless communication module of the optical imaging system.

Thus, the optical imaging system may be operated autarkic with respect to communication without the need for a communication or data connection cable within the surgical environment.

According to a further embodiment, the housing is adapted for receiving the image processing unit.

Thus, the optical imaging system may be operated autarkic with respect to image processing without the need for an extensive exchange of data between the optical imaging components in the housing within the surgical environment and external components.

According to a further embodiment, the first object is an x-ray imaging device and the first object tracking unit is an x-ray imaging device tracking unit, wherein the image processing unit is adapted to receive an x-ray image taken by the x-ray imaging device and to spatially correlate an x-ray image taken by the x-ray imaging device with the relative spatial position and orientation of the x-ray imaging device tracking unit and of the second object tracking unit with respect to each other.

Thus, it is possible to determine the spatial position and orientation of the x-ray imaging device with respect to the second object. This will also allow providing a correlation on the one hand of the relative position of the x-ray imaging device and the second object with respect to each other based on optical imaging, and on the other hand an x-ray image taken by said x-ray imaging device based on x-ray imaging.

According to a further embodiment, the x-ray imaging device tracking unit comprises a fiducial marker arrangement having a unique spatial projection allowing determining a spatial position and orientation of the x-ray imaging device with respect to an x-ray source providing x-rays for imaging upon x-ray imaging.

Thus, a spatial repositioning and reorientation between the x-ray source and e.g., an x-ray imaging device may be determined. When using a c-arm device, the x-ray source is located remote from the x-ray imaging device but connected through a longer c-arm. It cannot be excluded that that the c-arm connection deforms so that the spatial position and orientation, respectively of the x-ray source and the x-ray imaging device changes, which may lead to an image, which looks different from expected. The fiducial marker arrangement being mounted with respect to the x-ray imaging device in a reproducible spatial position and orientation may serve, if imaged through exposure to the x-ray source, to re-calculate the correct position and orientation of the x-ray source and the x-ray imaging device with respect to each other, even if the c-arm connection deforms. The fiducial marker arrangement may also be used for compensating distortions in imaging, in particular when using intensifier c-arm devices, and detection of mirrored x-ray images. The same applies for a fiducial marker arrangement which may be provided at the table where the patient is positioned on, which then allows determination of a positional and orientation deviation of the table and the x-ray source and the x-ray imaging device, respectively.

According to a further embodiment, the second object to be tracked is at least one of an implant and a tool.

Thus, an implant or a tool or even both may be tracked by the optical imaging system.

According to a further embodiment, the reference object is at least one of a tool and a patient's anatomy.

Thus, a tool or a patient's anatomy or even both may be tracked by the optical imaging system.

According to a further embodiment, the navigation system further comprises a display device being capable of displaying the determined relative spatial position and orientation of the first object tracking unit, e.g., an x-ray imaging device tracking unit and of the second object tracking unit with respect to each other. The second object may be displayed as a real image of the second object, or as an augmented second object or even both.

Thus, the imaged objects to be tracked may be displayed in a correct spatial position and orientation with respect to each other. This is not limited to a first and second object but may also include plurality of objects.

According to a further embodiment, the display device is further capable of displaying an x-ray image taken by the x-ray imaging device together with the determined relative spatial position and orientation of an x-ray imaging device tracking unit and of the second object tracking unit with respect to each other.

Thus, the display device can illustrate for a surgeon the relative position of the x-ray imaging device and the second object determined based on optical imaging and an x-ray image taken by the x-ray imaging device based on x-ray imaging. The second object may be a tool, so that a surgeon may see a tool, either as real image of the tool from an imaging procedure or an augmented tool based on the determined position and orientation of the unique pattern and an illustration of the identified tool from a database. The displayed tool may be displayed in an overlay illustration, so that a surgeon may see the tool in a position and orientation with respect to an x-ray image. The aforementioned description mutatis mutandis also applied for an implant, an anatomy or other objects.

According to a further embodiment, the display device is designed as a headset to be carried by a surgeon, which headset is adapted for augmenting the determined relative spatial position and orientation of the first object tracking unit, e.g., an x-ray imaging device tracking unit and of the second object tracking unit, e.g., a tool tracking unit, with respect to each other in real time with a view applied by said surgeon.

Thus, the correct spatial position and orientation of the objects to be tracked with respect to each other may be augmented to a real view of the surgeon. The surgeon may virtually add further objects, e.g., implants or tools and may immediately recognize whether the objects are in the intended spatial position and orientation with respect to each other. In order to determine a real viewing point for the x-ray image and the e.g., overlaid tool, corresponding to the actual surgeon position during surgery, the headset may also be considered as an object having an object tracking unit. The headset for this purpose may be equipped with a respective unique optical pattern allowing determination of the relative spatial position and orientation with respect to the other objects. This may allow determination of the actual viewing point and further allow real view display of the x-ray image with the overlaid tool and/or implant and/or anatomy.

According to a further embodiment, the display device is further capable of augmenting an x-ray image taken by the x-ray imaging device together with the determined relative spatial position and orientation of the x-ray imaging device tracking unit and of the second object tracking unit with respect to each other.

Thus, the display device can augment for a surgeon as real view the x-ray image taken by the x-ray imaging device and the relative spatial position and orientation of the x-ray imaging device and the second object.

According to an embodiment, there is provided an optical imaging system for being positioned in a surgical environment, the optical imaging system comprises as a first optical imaging unit a first camera with a lens having a wide opening angle, and as a second optical imaging unit a second camera with a lens having a wide opening angle, a mechanical interface for coupling the optical imaging system to an operating table within said surgical environment, wherein the first camera and the second camera are arranged back-to-back.

Thus, a simple optical imaging system may be provided, which allows imaging taking over a wide-angle imaging area. Back-to-back mounting allows a defined geometry of the cameras, which may simplify calibration of the imaging areas of the single cameras with respect to each other. The mechanical interface allows mounting to an operating table and may include an adapted for a table system or any other known geometry being able to be fixed to an operating table. Instead of back-to-back mounting, a triangular mounting of three cameras may be provided. Wide opening angle may be understood as a horizontal opening angle (or generally an opening angle in a first direction) of e.g., at least 120°, in particular 150°, in particular 180°, in particular larger than 180°, in particular between 180° and 190°. Wide opening angle may be understood as a vertical opening angle (or generally an opening angle in a second direction orthogonal to the first direction) of e.g., at least 120°, in particular 150°, in particular 180°, in particular larger than 180°, in particular between 180° and 190°. The opening angle in horizontal direction and in vertical direction may be the same.

According to an embodiment, there is provided an optical imaging system for being positioned in a surgical environment, the optical imaging system comprises as a first optical imaging unit a first camera with a lens having a wide opening angle, and as a second optical imaging unit a second camera with a lens having a wide opening angle, a mechanical interface for coupling the optical imaging system to a patient's anatomy within said surgical environment, wherein the first camera and the second camera are arranged back-to-back.

Thus, a simple optical imaging system may be provided, which allows imaging taking over a wide-angle imaging area. Back-to-back mounting allows a defined geometry of the cameras, which may simplify calibration of the imaging areas of the single cameras with respect to each other. The mechanical interface allows mounting to a patient's anatomy and may include a portion with a pin or any other known geometry being able to be fixed to a patient's anatomy. Instead of back-to-back mounting, a triangular mounting of three cameras may be provided. Wide opening angle may be understood as a horizontal opening angle (or generally an opening angle in a first direction) of e.g., at least 120°, in particular 150°, in particular 180°, in particular larger than 180°, in particular between 180° and 190°. Wide opening angle may be understood as a vertical opening angle (or generally an opening angle in a second direction orthogonal to the first direction) of e.g., at least 120°, in particular 150°, in particular 180°, in particular larger than 180°, in particular between 180° and 190°. The opening angle in horizontal direction and in vertical direction may be the same.

According to a further embodiment, there is provided an optical imaging system for being positioned in a surgical environment, the optical system comprises at least a first optical imaging unit and a housing, wherein the optical imaging system has a continuous optical imaging area covering at least a unique optical pattern of a first object tracking unit, e.g. an x-ray imaging device tracking unit, and a unique optical pattern of a second object tracking unit within the surgical environment, wherein the housing comprises the mechanical interface for coupling the optical imaging system to a reference object within said surgical environment, wherein the housing comprises an optical image unit receiving interface for interchangeably receiving at least the first optical imaging unit for establishing a reproducible spatial position and orientation of the at least first optical imaging unit with respect to the optical imaging system. The first optical imaging unit may be the aforementioned first camera and the second optical imaging unit may be the afore mentioned second camera.

Thus, an optical imaging system may be provided with the properties as described above, even if provided as a separate device apart from the above describe navigation system.

According to a further embodiment, the housing is a single use housing comprising deformable housing sections, which deformable housing sections are adapted to modify essential housing parts upon a sterilization process, such that a reuse of the housing is prevented upon application of a sterilization process.

Thus, it may be avoided that a housing may be re-used. If against the intention to use the housing only once, the housing is sterilized, the housing may deform, which deformation may also include a change of essential properties required for operating the optical system. This may also include clouding of windows or optical lenses provided at the housing side to avoid re-use of the housing. The change may be generated by different impacts each resulting from a sterilization.

According to a further embodiment, there is provided a method comprising: optical imaging by an imaging system, mounted to a reference object in a reproducible spatial position and orientation thereto within a surgical environment, a unique optical marker on an x-ray imaging device tracking unit mounted to an x-ray imaging device in a defined spatial position and orientation with respect to the optical imaging system; optical imaging by the imaging system, mounted to said reference object in a reproducible spatial position and orientation thereto within a surgical environment, a unique optical marker on the second object tracking unit mounted to a second object in a defined spatial position and orientation with respect to the optical imaging system; x-ray imaging of the surgical environment by the x-ray imaging device; correlating optical imaging the unique optical marker on the x-ray imaging device tracking unit, optical imaging the unique optical marker on the second object tracking unit and x-ray imaging of the surgical environment; determining a position and orientation of the x-ray imaging device, a position and orientation of the second object, and the x-ray image taken by the x-ray imaging device based on the correlating.

Thus, a method can be provided which corresponds to the navigation system as described above and expresses the operation of the navigation system. Steps S20, S30 and S40 may carried out synchronous, i.e., at the same time.

According to a further embodiment, wherein correlating optical imaging the unique optical marker on the x-ray imaging device tracking unit, optical imaging the unique optical marker on the second object tracking unit and x-ray imaging of the surgical environment; and determining a position and orientation of the x-ray imaging device, a position and orientation of the second object, and the x-ray image taken by the x-ray imaging device based on the correlating is carried out on an image processing unit provided on the optical imaging system.

According to a further embodiment, a method for conducting a surgical procedure is provided. A method according to this embodiment, may include the steps of placing an optical imaging system proximate to a target anatomy of a patient, obtaining a spatial position and orientation of a first object within a first optical imaging area using a first optical imaging unit, obtaining a spatial position and orientation of a second object within a second optical imaging area using a second optical imaging unit, and guiding a placement of the second object with reference to the target anatomy by determining a relative spatial position and orientation of the first object and the second object with respect to each other based on optical images taken by the optical imaging system. The optical imaging system may include the first optical imaging unit with the first optical imaging area and the second optical imaging unit with the second optical imaging area.

Continuing in accordance with this embodiment, the second object may be any of an implant or surgical tool. The first optical imaging unit and the second optical imaging unit may each have a camera with a lens having an opening angle of at least 180 degree (half sphere). The first optical imaging unit and the second optical imaging unit may be arranged back-to-back such that the first optical imaging area and the second optical imaging area together form an optical imaging area of 360 degrees (full sphere). The step of guiding the placement of the implant or surgical tool with reference to the target anatomy may be performed intraoperatively.

Continuing in accordance with this embodiment, the first object may be an x-ray imaging device. The method may further include the steps of receiving an x-ray image from the x-ray imaging device and spatially correlating the x-ray image with the relative spatial position and orientation of the x-ray imaging device and of the implant or surgical tool with respect to each other.

Continuing in accordance with this embodiment, the first object may include a first object tracking unit comprising a unique optical pattern for determining a spatial position and orientation of the first object upon optical imaging with respect to the imaging system. The implant or surgical tool may comprise a second object tracking unit which may include a unique optical pattern for determining a spatial position and orientation of the second object upon optical imaging with respect to the imaging system.

Thus, a surgical procedure for positioning an implant or a surgical tool at target surgical site using an optical imaging system may take place without the need for an external imaging device in the surgical surrounding.

It should be noted that the above-described embodiments may also be combined and in a combined form provide a synergetic technical effect and synergetic benefits which go beyond the sum of the single technical effects and benefits.

### BRIEF DESCRIPTION OF THE FIGURES

The invention will be described by way of the following drawings:

FIG. 1: illustrates a schematic view of an exemplary embodiment of a navigation system in a surgical surrounding.

FIG. 2: illustrates an exemplary embodiment of an imaging system for a navigation system in a cross-sectional side view.

FIG. 3: illustrates an exemplary embodiment of an imaging system for a navigation system in a cross-sectional top view.

FIG. 4: illustrates a side view of an exemplary application scenario in a surgical environment for the navigation system.

FIG. 5: illustrates a side view of a further exemplary application scenario in a surgical environment for the navigation system.

FIG. 6: illustrates a side view of another exemplary application scenario in a surgical environment for the navigation system.

FIG. 7: illustrates a side view of another further exemplary application scenario in a surgical environment for the navigation system.

FIG. 8: illustrates a schematic view of an exemplary embodiment of a navigation method.

It should be noted that same or similar reference numerals illustrate same or similar components. Along these Figures exemplary embodiments of the invention will be described as follows.

### DETAILED DESCRIPTION OF EXEMPLARY EMBODIMENTS

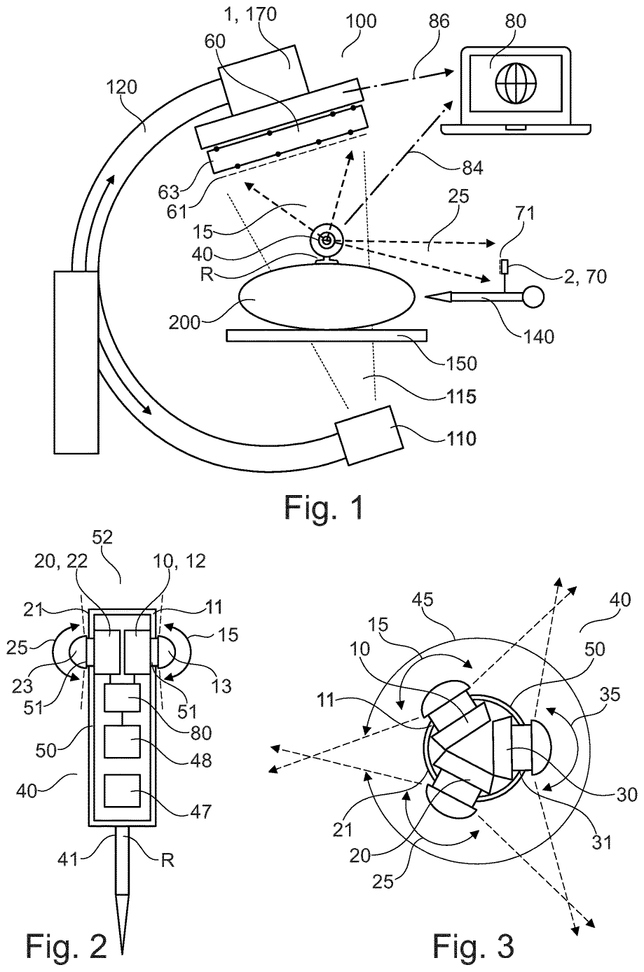

FIG. 1 illustrates an exemplary embodiment of a navigation system in a surgical surrounding. FIG. 1 illustrates a setup of the surgical environment with a patient or patient's anatomy 200 laying on a table 150 of an operation room. Additionally, a c-arm x-ray device 120 is provided having an x-ray source 110 as well as an x-ray imaging device 170. The x-ray imaging device is provided with an x-ray device tracking unit 60 having a unique optical pattern 61, representative for a spatial position and orientation of the x-ray imaging device 170. As the optical pattern 61 is unique, it is possible to determine from an image taken from the unique optical pattern, based on the evaluation of pattern details, their position with respect to each other and from the image distortion resulting from the viewing angle and perspective to the spatial position and orientation of the unique optical pattern. If a corresponding object is positioned in a defined spatial position and orientation with respect to the unique optical pattern, also the spatial position and orientation of the object can be determined. As a consequence, the tracking unit 60 with the unique optical pattern 61 mounted to the x-ray imaging device 170 in a defined manner allows determination of the spatial position and orientation of the x-ray imaging device 170. It should be noted that this generally applies to any object 1, having mounted a unique optical pattern 61 thereto.

The navigation system 100 comprises an optical imaging system 40 which can be mounted to a reference object R in a defined spatial position and orientation. The optical imaging system is positioned in the surgical environment, which can be considered as the space where all objects immediately related to the surgical procedure are located, e.g. the patient 200, the operating table 150, implants 130, tools 140, x-ray imaging devices 170 and their respective tracking units 60, 70 with their unique optical patterns 61, 71. Beside the patient 200 itself, relevant objects are a first object 1 with a first object tracking unit 60, here the x-ray imaging device 170, and a second object 2 with a second object tracking unit 70. The related unique optical patterns are the optical pattern 61 of the first object tracking unit 60 for the first object 1, and the optical pattern 71 for the second object tracking unit 70 for the second object. The optical imaging system 40 has a wide-angle imaging area 45, which is capable of imaging both, the first optical pattern 61 allocated to the first object 1 and the second optical pattern 71 allocated to the second object 2. It should be noted that the number of objects and respective unique optical patterns is not limited, and that the unique optical pattern may also carry information for identification of the allocated object. The optical imaging system 40 may have a plurality optical imaging units 10, 20. The imaging area 15 of the first optical unit 10 and the imaging area 25 of the second imaging unit 20 may abut or overlap so as to form a continuous imaging area 45. The illustrated embodiment shows imaging of the first object 1 with the first imaging unit 10 with a first imaging area 15 and imaging of the second object 2 with the second imaging unit 20 with a second imaging area 25. It should be noted that in case the orientation of the optical imaging system 40 is modified, it may also be possible to image the first object 1 and the second object 2 by only the first imaging unit 10 or only the second imaging unit 20. As both imaging areas abut or overlap, the image processing may access to the entire imaging area composed of the first imaging area 15 and the second imaging area. The same applies for three or more imaging units, as will be described with respect to FIG. 3. The x-ray imaging device 170 may provide the x-ray image data through a data link 86 to the image processing unit 80. Likewise, the optical imaging system 40 may provide the optical image data through a respective data link 84 to the image processing unit 80, which image processing unit may correlate the x-ray image data and the optical image data. By determining the spatial position and orientation of the first object 1, here the x-ray imaging device, and the second object 2 with respect to each other, the x-ray image taken by the x-ray imaging device may be correlated also to the position and orientation of the second object 2. This allows to determine a spatial relationship between the x-ray image and the second object, although the second object 2 does not need to be in the radiation area 115 of the x-ray source 110, and thus does not need to be imaged by the x-ray imaging device 60. Usually, the second object 2, e.g., an implant 130 or a tool 140 are moved during surgery. As the optical imaging system 40 may track the spatial position and orientation of the second object 2 by imaging its allocated unique optical pattern 71, repositioning of the tool 140 or implant 130 does not require a new x-ray imaging, which saves radiation exposure to the patient 200.

It can be summarized that the navigation system 100 may be used for intraoperatively tracking instruments or implants, patient's anatomy, and c-arms, augmented reality or mixed reality or virtual reality headsets/visualization devices and other objects. As the imaging system 40 is provided in the surgical environment, where usually only the surgeon's hands are present, but not the surgeon's entire body, the system has very small footprint, short setup time, and is reliable and easy to use. An exemplary navigation system comprises the following components: A wide angle imaging unit or camera group, which may cover e.g., 360 degrees (full sphere). The camera group may be considered as a centerpiece of the system and allows tracking optical markers 61, 71 attached to instruments 140 or implants 130, to the patient 200, and/or to the c-arm 120 and c-arm components 110, 170. The camera group may have the following properties: The camera group may comprise two imaging units or cameras 10, 20 each having a so-called fish-eye lens (with an ~180 degree opening angle, and e.g., ~10 cm minimum object distance, and e.g., a fixed focal length). The two camera units 10, 20 of the optical imaging system 40 may be mounted back-to-back, each with a high-resolution imaging sensor so as to allow capturing the entire (360 degree) surroundings of the camera. The optical system 40 thus may have small size and be lightweight. The optical imaging system 40 with the camera group may have e.g., a weight of less than 200 grams and a small size of e.g., 15 cm×3 cm×3 cm.

The Navigation system 100 may further comprise: A so called c-arm tracker and calibration phantom. The c-arm tracker may have a group of fiducial markers 63 having a unique projection for each spatial position and orientation. The fiducial markers 63 may be attached to the x-ray image detector 170 of the c-arm 120 during the entire procedure and may serve for two purposes: As a first purpose a calibration of the c-arm projection geometry. For this purpose, the c-arm tracker may contain fiducial marker, e.g., beads, in a 3D arrangement which are visible in the x-ray images and allow computation of c-arm projection geometry parameters such as focal point position, pixel size, etc. As a further purpose, the fiducial marker 63 may also allow tracking of the c-arm position. The c-arm tracker 60 may also comprise optical markers/patterns 61 attached to it which allow tracking of the c-arm position with respect to the 360-degree camera. The optical markers 61 may be placed on the tracker in a fixed and accurately known position with respect to the fiducial marker and may be used for computing the projection geometry. The c-arm tracker may be designed as an exchangeable unit and may be re-usable and may be draped during surgery. A special drape with a transparent part adapted to the c-arm tracker may be used to make the optical tracking pattern 61 visible under the drape without reflections or optical errors introduced by wrinkles in the drape. Alternatively, the optical pattern 61 may be designed to be sterile and may be designed as a re-usable unit or a not-re-usable unit and can be attached to the re-usable part of the tracker through the drape.

The navigation system 100 may further have instrument/implant tracker 70. The instrument/implant trackers 70 may have optical patterns 71 attached to instruments 140 or implants 130 allowing accurate tracking of the position and orientation of the respective instrument 140/implant 130 with respect to the 360-degree camera group.

The navigation system may further have a host computer including an image processing unit 80. The host computer may have at least the following functionalities: Capturing of x-ray images from the c-arm 120, in particular the imaging device of the c-arm via a communication connection 86. The host computer may use a frame-grabber to capture x-ray images from the c-arm. The host computer may automatically detect when a new x-ray image is taken. The host computer, in particular the image processing unit may be in wired (or optionally may have a wireless) communication 84 with the 360-degree camera. The host computer, in particular the image processing unit may be used for tracking computation. The host computer, in particular the image processing unit 80 may receive an image stream or alternatively a feature stream from the 360-degree camera and may perform tracking computations for determining the position of c-arm 120, 170 and instruments 140/implants 130 with respect to the optical imaging system 40 with the camera group. Optionally the entire tracking computation or part of the tracking computation is performed on the optical imaging system 40. In this case, the image processing is provided in the unit of the optical imaging system 40. This then reduces the bandwidth requirements for communication between optical imaging system 40 and a host computer.

The navigation system 100 may have the following value proposition: The system may enable navigated trauma/foot & ankle surgery and therefore may increase accuracy and repeatability of procedures, increases confidence of surgeons, and reduces x-ray-radiation. The system can be used for multiple different clinical use-cases without needing to develop and test a reference body specifically for each use-case. The system allows for live c-arm tracking. The position of the c-arm with respect to a target position can be shown with live updates to the user and therefore c-arm positioning is simplified. This directly translates to a reduction of radiation because the number of test shots needed for positioning the c-arm is reduced. The system allows for live implant tracking if either the camera or a tracking pattern is attached to the implant. Instruments only need to be equipped with tracking patterns, no active control electronics and control cable is needed at the instrument. Compared to navigation systems using a stereo camera, the proposed system has the following advantages: The system has a much smaller footprint. The camera is designed to be placed in the sterile field and is much smaller than a stereo camera. This saves space in the operation room, storage space in the hospital, and makes the system more user-friendly and reduces line-of-sight problems

FIG. 2 illustrates an exemplary embodiment of an imaging system 40 for a navigation system 100 in a cross-sectional side view. The optical imaging system 40 in this embodiment has a first optical imaging unit 10 and a second optical imaging unit 20. Both optical imaging units 10, 20 are mounted back-to-back. Each of the optical imaging units 10, 20 has a large opening angle and thus a wide-angle image area 15, 25 of about 180 degrees (each has a half sphere). The first optical image unit 10 has a mechanical interface 11 to be received in a respective counter interface 51 of a housing 50 of the optical imaging system 40. The first optical imaging unit 10 further has a camera 12 and a lens 13 of camera 12 allowing a wide-angle imaging area 15. Likewise, the second optical image unit 20 has a mechanical interface 21 to be received in a respective counter interface 51 of a housing 50 of the optical imaging system 40. The second optical imaging unit 20 further has a camera 22 and a lens 23 of camera 22 allowing a wide-angle imaging area 25. Both optical imaging units 10, 20 may be coupled to the optical imaging system 40 via a respective mechanical interface of the optical imaging system 40. This interface may be realized as interface 51 of the housing 50 for receiving both optical imaging units 10, 20.

The optical imaging system 40 may further comprise a power supply, which may be a cordless power supply in form of a battery or a rechargeable battery, if provided within the housing 50. Further, the optical imaging system 40 may have a communication module 48, which may be a wireless communication module when provided in the housing 50.

The housing 50 may have a deformable housing section 52, which may deform upon a sterilization process, be it a thermal sterilization or a physical sterilization or a chemical sterilization. Deformation here means that a housing part is modified upon sterilization so that this housing part may avoid re-use of the optical imaging system 40 without exchanging at least the housing, or also the entire optical imaging system 40 including the housing 50. Deformation may also include clouding of optical elements provided in the housing, e.g., windows through which optical imaging takes place by the imaging units 10, 20. Thus, a sterile, single-use housing 50, may be provided. The imaging units 10, 20 with the camera may be inserted into a sterile, single-use housing 50. This allows use of the camera in the sterile field during surgery. The housing 50 may be discarded after surgery, the optical imaging units 10, 20, 30 with the cameras may be re-usable. The optical imaging system 40, in particular the housing 50 thereof may be provided with a mechanical interface 51. The imaging system 40 (or the sterile housing 50) has a mechanical interface 41 to allow easy attachment to e.g., pins inserted into bones of the patient 200, instrumentation (e.g., gamma targeting arm) or the operation room table 150. Optionally the optical imaging system 40 has onboard power 47, onboard image processing 80, onboard sensing, and/or onboard wireless communication capabilities 47. The imaging system 40 might use a battery for power supply, additional sensors such as for example an IMU for detecting the direction of gravity or a microphone for voice control, advanced image processing capabilities to perform onboard image processing, and wireless communication capabilities to exchange data with a host computer.

FIG. 3 illustrates an exemplary embodiment of an imaging system for a navigation system in a cross-sectional top view. The illustrated imaging system 40 in FIG. 3 has three optical imaging units 10, 20, 30. The description of FIG. 2 applies likewise also for a system 40 with three imaging units 10, 20, 30. The system in FIG. 3 illustrates a mechanical interface 11 of first imaging unit 10, a mechanical interface 21 of second imaging unit 20 and a mechanical interface 31 of third imaging unit 30. Likewise, each of the units 10, 20, 30 has a camera with a respective lens. The imaging areas may be somewhat differently compared to what is illustrated in FIG. 2, as each unit 10, 20, 30 has only to cover one third of the entire sphere in order to have a full sphere imaging area in total. This is illustrated as the respective imaging areas 15, 25 and 35 for the first imaging unit 10, the second imaging unit 20 and the third imaging unit 30, respectively. All three imaging areas 15, 25 and 35 together form the continuous imaging area 45. In case more than three imaging units are provided, the imaging areas may be adapted according to need.

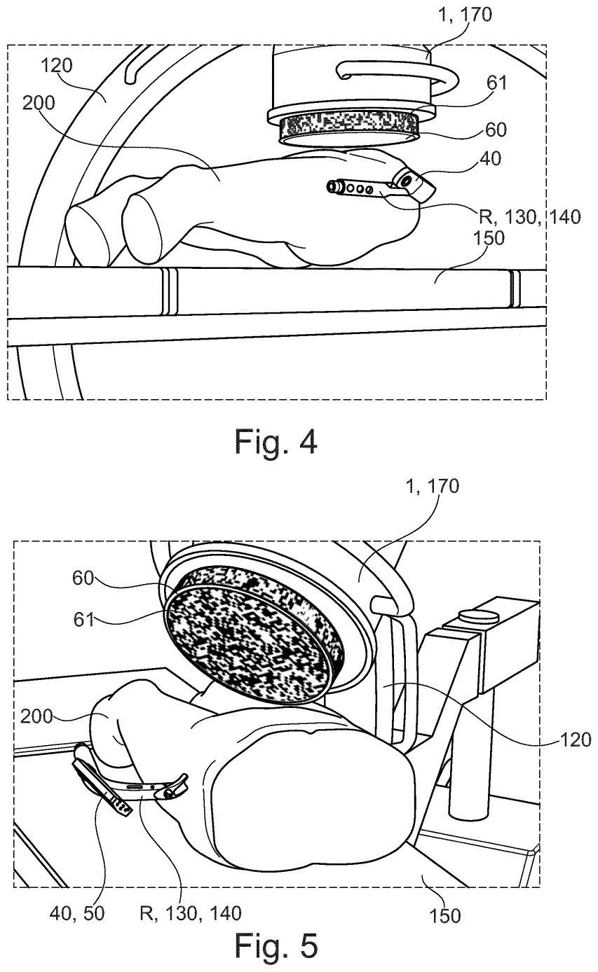

FIG. 4 illustrates an exemplary application scenario in a surgical environment for the navigation system. In FIG. 4 it is illustrated that a patient's anatomy 200 is positioned on an operation room table 150. The c-arm 120 is positioned so as to take an x-ray image by activating the x-ray source 110 and taking the respective image with the x-ray imaging device 170, which may also be considered as a first object 1 in a surgical environment to be tracked. The x-ray imaging device 170 here has a unique optical pattern 61, and here serves as a tracking unit. In the surgical environment there is also provided the optical imaging system 40 in form of a camera group in a housing. This optical imaging system 40 in FIG. 4 is mounted to the insertion handle of an implant 130, so as to form a reference object R, as described above. Instead of an implant 130, also a tool may be used.

FIG. 5 illustrates a further exemplary application scenario in a surgical environment for the navigation system, which corresponds to what is illustrated in FIG. 4, however from a different viewing point. Therefore, the description of FIG. 4 likewise applies for FIG. 5.

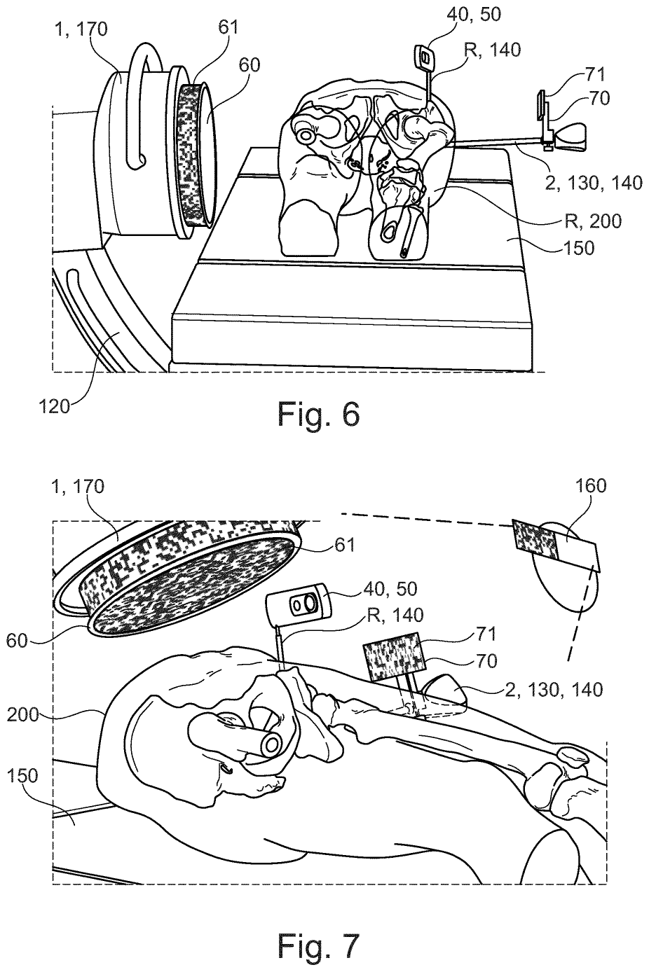

FIG. 6 illustrates an exemplary application scenario in a surgical environment for the navigation system. In FIG. 6 it is illustrated that a patient's anatomy 200 is positioned on an operation room table 150. The c-arm 120 is positioned so as to take an x-ray image by activating the x-ray source 110 (not shown) and taking the respective image with the x-ray imaging device 170, which may also be considered as a first object 1 in a surgical environment to be tracked. The x-ray imaging device 170 also here has a unique optical pattern 61 and serves as a tracking unit. Within the surgical environment there is also provided the optical imaging system 40 in form of a camera group in a housing 50. This optical imaging system 40 in FIG. 6 is mounted to a tool, e.g., in form of a pin 140, which is fixed to the patient anatomy, so as to form a reference object R, as described above. FIG. 6 also illustrates a further tool 140, which may e.g., have coupled an implant 130. The tool 140, which may be considered as a second object 2 to be tracked, has a tracking unit 70, mounted in defined spatial position and orientation with respect to the second object 2 to be tracked. The tracking unit 70 has a unique optical pattern 71, which may be imaged by the imaging system 40. The imaging system 40 likewise may image the optical pattern 61 of the x-ray imaging device 170. Both the imaging of the x-ray imaging device 170 and the tool 140, each represented by its respective unique optical pattern 61, 71, as well as the x-ray image taken by the x-ray imaging device 170 are correlated by the imaging processing unit 80, as described above.

FIG. 7 illustrates a further exemplary application scenario in a surgical environment for the navigation system, which corresponds to what is illustrated in FIG. 6, however from a different viewing point. Therefore, the description of FIG. 6 likewise applied for FIG. 7.

FIG. 7 further illustrates a display device 160 being capable of displaying the determined relative spatial position and orientation of the first object tracking unit 60, here the x-ray imaging device tracking unit, and of the second object tracking unit 70 with respect to each other, and thus also the relative spatial position and orientation of the first and second object 1, 2. The display device may be capable of displaying an x-ray image taken by the x-ray imaging device 170 together with the determined relative spatial position and orientation of an x-ray imaging device tracking unit 60 and of the second object tracking unit 70 with respect to each other. Here the display device is designed as a head set 160 carried by a surgeon. The headset may augment an x-ray image taken by the x-ray imaging device 170 together with the determined relative spatial position and orientation of the x-ray imaging device tracking unit 60 representing the first object 1, here the x-ray imaging device 170, and of the second object tracking unit 70 representing the second object 2 with respect to each other.

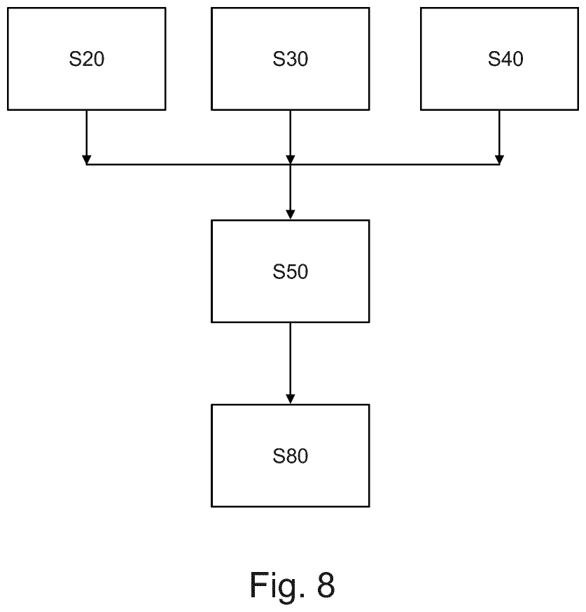

FIG. 8 illustrates an exemplary embodiment of a navigation method. The method comprises: optical imaging S20 by an imaging system 40, mounted to a reference object R in a reproducible spatial position and orientation thereto within a surgical environment, a unique optical marker 61 on an x-ray imaging device tracking unit 60 mounted to an x-ray imaging device 170 in a defined spatial position and orientation with respect to the optical imaging system 40; optical imaging S30 by the imaging system 40, mounted to said reference object R in a reproducible spatial position and orientation thereto within a surgical environment, a unique optical marker 71 on the second object tracking unit 70 mounted to a second object 2 in a defined spatial position and orientation with respect to the optical imaging system 40; x-ray imaging S40 of the surgical environment by the x-ray imaging device 170; correlating S50 optical imaging S20 the unique optical marker 61 on the x-ray imaging device tracking unit 60, optical imaging S30 the unique optical marker 71 on the second object tracking unit 70 and x-ray imaging S40 of the surgical environment; determining S80 a position and orientation of the x-ray imaging device 170, a position and orientation of the second object 2, and the x-ray image taken by the x-ray imaging device 170 based on the correlating S50. Steps S20, S30 and S40 may be carried out synchronous, i.e., at the same time. Steps S20, S30 and S40 may also be carried out in sequence, as long as the position of the objects with respect to each other does not change, or the change is tracked, and a compensation calculation is carried out. Correlating S50 optical imaging S20 the unique optical marker 61 on the x-ray imaging device tracking unit 60, optical imaging S30 the unique optical marker 71 on the second object tracking unit 70 and x-ray imaging S40 of the surgical environment; and determining S80 a position and orientation of the x-ray imaging device 170, a position and orientation of the second object 2, and the x-ray image taken by the x-ray imaging device 170 based on the correlating S50 is carried out on an image processing unit 80 provided on the optical imaging system 40.

### REFERENCE LIST

R reference object within surgical environment
1 first object within surgical environment
2 second object within surgical environment
10 first imaging unit
11 mechanical interface of first imaging unit
12 camera of first optical imaging unit
13 lens of camera of first optical imaging unit
15 first optical imaging area of first imaging unit
20 second imaging unit
21 mechanical interface of second imaging unit
22 camera of second optical imaging unit
23 lens of camera of second optical imaging unit
25 second optical imaging area of second imaging unit
30 third imaging unit
35 third optical imaging area of third imaging unit
40 imaging system
41 mechanical interface of imaging system
45 continuous optical imaging area
47 cordless power supply of the optical imaging system
48 wireless communication module of the optical imaging system
50 housing of imaging system
51 receiving interface of housing for optical imaging unit(s)
52 deformable section of housing
60 first object tracking unit
61 unique optical pattern of first object tracking unit
63 fiducial marker arrangement of x-ray imaging device/first object tracking unit
70 second object tracking unit
71 unique optical pattern of second object tracking unit
80 imaging processing unit
84 image data transfer from imaging system to image processing unit
86 image data transfer from x-ray imaging device to image processing unit
100 navigation system
110 x-ray source
115 x-ray radiation sector
120 c-arm
130 implant
140 tool
150 operating table
160 display device, augmented reality glasses
170 x-ray imaging device
200 anatomy
S10 mounting an optical imaging system
S20 optical imaging a unique optical marker on a first object tracking unit
S30 optical imaging a unique optical marker on the second object tracking unit
S40 x-ray imaging of the second object
S50 correlating optical imaging S20, optical imaging S30 and x-ray imaging S40
S80 determining position and orientation of first object with respect to second object

1. A navigation system for intraoperatively tracking objects during surgery, the system comprising:
an optical imaging system with a first optical imaging unit with a first optical imaging area and a second optical imaging unit with a second optical imaging area, and with a mechanical interface for coupling the imaging system to a reference object within a surgical environment;
a first object tracking unit to be coupled to a first object, representing a spatial position and orientation of the first object within a surgical environment;
a second object tracking unit to be coupled to a second object, representing a spatial position and orientation of the second object within a surgical environment;
an image processing unit;
wherein the first optical imaging unit and the second optical imaging unit are arranged with respect to each other such that the first optical imaging area and the second optical imaging area form an optical imaging area,
wherein the first object tracking unit comprises a unique optical pattern allowing determining a spatial position and orientation of the first object upon optical imaging with respect to the imaging system;
wherein the second object tracking unit comprises a unique optical pattern allowing determining a spatial position and orientation of the second object upon optical imaging with respect to the imaging system;
wherein the optical imaging area covering at least the unique optical pattern of the first object tracking unit and the unique optical pattern of the second object tracking unit;
wherein the imaging processing unit is adapted to determine the relative spatial position and orientation of the first object tracking unit and of the second object tracking unit with respect to each other based on optical images taken by the optical imaging system.

2. The navigation system according to claim 1, wherein the first optical imaging unit and the second optical imaging unit each have a camera with a lens having an opening angle of at least 180 degree (half sphere), wherein the first optical imaging unit and the second optical imaging unit are arranged back to back so that the first optical imaging area and the second optical imaging area together form the optical imaging area of 360 degrees (full sphere).

3. The navigation system according to claim 1, wherein the mechanical interface of the optical imaging system is adapted for establishing a reproducible spatial position and orientation of the optical imaging system with respect to the reference object within the surgical environment.

4. The navigation system according to claim 1, wherein the imaging processing unit is adapted to determine the relative spatial position and orientation of the reference object, the first object tracking unit and of the second object tracking unit with respect to each other based on optical images taken by the optical imaging system.

5. The navigation system according to claim 1, wherein the optical imaging system comprises a housing, wherein the housing comprises the mechanical interface for coupling the optical imaging system to the reference object within a surgical environment, wherein the housing comprises an optical image unit receiving interface for interchangeably receiving at least one optical imaging unit of the optical imaging system for establishing a reproducible spatial position and orientation of the at least one optical imaging unit with respect to the optical imaging system, wherein the housing is a single use housing comprising deformable housing sections, which deformable housing sections are adapted to modify essential housing parts upon a sterilization process, such that a reuse is prevented upon application of a sterilization process.

6. (canceled)

7. The navigation system according to claim 5, wherein the housing is adapted for receiving at least one of a cordless power supply of the optical imaging system, a wireless communication module of the optical imaging system and the image processing unit.

8-9. (canceled)

10. The navigation system according to claim 1, wherein the first object is an x-ray imaging device and the first object tracking unit is an x-ray imaging device tracking unit, wherein the imaging processing unit is adapted to receive an x-ray image taken by the x-ray imaging device and to spatially correlate an x-ray image taken by the x-ray imaging device with the relative spatial position and orientation of the x-ray imaging device tracking unit and of the second object tracking unit with respect to each other, wherein the x-ray imaging device tracking unit comprises a fiducial marker arrangement having a unique spatial projection allowing determining a spatial position and orientation of the x-ray imaging device with respect to an x-ray source providing x-rays for imaging upon x-ray imaging.

11-13. (canceled)

14. The navigation system according to claim 1, further comprising a display device being capable of displaying the determined relative spatial position and orientation of the first object tracking unit and of the second object tracking unit with respect to each other.

15. (canceled)

16. The navigation system according to claim 14, wherein the display device is designed as a headset to be carried by a surgeon, which headset is adapted for augmenting the determined relative spatial position and orientation of the first object tracking unit and of the second object tracking unit with respect to each other in real time with a view applied by said surgeon.

17. The navigation system according to claim 14, wherein the display device is further capable of augmenting an x-ray image taken by the x-ray imaging device together with the determined relative spatial position and orientation of the x-ray imaging device tracking unit and of the second object tracking unit with respect to each other.

18. An optical imaging system for being positioned in a surgical environment, the optical imaging system comprises:
as a first optical imaging unit a first camera with a lens having a wide opening angle, and
as a second optical imaging unit a second camera with a lens having a wide opening angle,
a mechanical interface for coupling the optical imaging system to at least one of an operating table within said surgical environment and a patient's anatomy within said surgical environment,
wherein the first camera and the second camera are arranged back-to-back.

19. (canceled)

20. An optical imaging system for being positioned in a surgical environment, the optical system comprising:
at least a first optical imaging unit
wherein the optical imaging system has an optical imaging area covering at least a unique optical pattern of a first object tracking unit and a unique optical pattern of a second object tracking unit within the surgical environment;
wherein the optical imaging system comprises a mechanical interface for coupling the optical imaging system to a reference object within said surgical environment.

21. The optical imaging system according to claim 20, wherein the optical imaging system comprises a first optical imaging unit with a first optical imaging area and a second optical imaging unit with a second optical imaging area, wherein the first optical imaging unit and the second optical imaging unit are arranged with respect to each other such that the first optical imaging area and the second optical imaging area form the optical imaging area.

22. The optical imaging system according to claim 21, wherein the first optical imaging unit and the second optical imaging unit each have a camera with a lens having an opening angle of at least 180 degree (half sphere), wherein the first optical imaging unit and the second optical imaging unit are arranged back to back so that the first optical imaging area and the second optical imaging area together form the optical imaging area of 360 degrees (full sphere).

23. The optical imaging system according to claim 18, further comprising a housing, wherein the housing comprises an optical image unit receiving interface for interchangeably receiving at least the first optical imaging unit for establishing a reproducible spatial position and orientation of the at least first optical imaging unit with respect to the optical imaging system.

24. The optical imaging system according to claim 23, wherein the housing is a single use housing comprising deformable housing sections, which deformable housing sections are adapted to modify essential housing parts upon a sterilization process, such that a reuse of the housing is prevented upon application of a sterilization process.

25-26. (canceled)

27. A method for performing a surgical procedure, the method comprising the steps of:
placing an optical imaging system proximate to a target anatomy of a patient, the optical imaging system having a first optical imaging unit with a first optical imaging area and a second optical imaging unit with a second optical imaging area;
obtaining a spatial position and orientation of a first object within the first optical imaging area using the first optical imaging unit;
obtaining a spatial position and orientation of a second object within the second optical imaging area using the second optical imaging unit, and
guiding a placement of the second object with reference to the target anatomy determining a relative spatial position and orientation of the first object and the second object with respect to each other based on optical images taken by the optical imaging system.

28-29. (canceled)

30. The method of claim 27, wherein the step of guiding the placement of the implant or surgical tool with reference to the target anatomy is performed intraoperatively.

31. (canceled)

32. The method of claim 27, further including the steps of receiving an x-ray image from the x-ray imaging device and spatially correlating the x-ray image with the relative spatial position and orientation of the x-ray imaging device and of the implant or surgical tool with respect to each other.

33. The method of claim 27, wherein the first object includes first object tracking unit comprising a unique optical pattern for determining a spatial position and orientation of the first object upon optical imaging with respect to the imaging system, the implant or surgical tool comprising a second object tracking unit comprises a unique optical pattern for determining a spatial position and orientation of the second object upon optical imaging with respect to the imaging system.
